(19) United States
(12) Patent Application Publication
(10) Pub. No.: US 2021/0338858 A1
(43) Pub. Date: Nov. 4, 2021

(54) Money Disinfectant Machine

(71) Applicant: Dilcia Granville, Nassau County, NY (US)

(72) Inventor: Dilcia Granville, Nassau County, NY (US)

(21) Appl. No.: 16/865,360

(22) Filed: May 3, 2020

(51) Int. Cl. A61L 2/10; G07G 1/00; A61L 2/04

(52) U.S. Cl. CPC A61L 2/10; G07G 1/0027; A61L 2202/26; A61L 2202/11; A61L 2202/14; A61L 2/04

(57) ABSTRACT

The present invention discloses an enhanced design of money disinfectant system which provides quality of high ratio of killing germs and is easily cleanable, closable, reusable, and offer the convenient option to disinfect and work smartly with money counting machines. The assembly is in form of a stainless material which combines Ultraviolet Light (UV) with a Heat element. The assembly is a handheld unit and can be added to many other money, related machines, to kill deadly viruses and germs by applying heat and/or UV during a short period time.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The invention relates to a kind of paper currency sterilizing apparatus and relates in particular to an ultraviolet and heating element sterilization system used for preventing the transfer of microbes from currency to human beings.

### Description of the Related Art

Currency has been widely circulated from the past through bills and coins as the most basic means of value that form the basis of the economic structure. The Money Disinfectant (MD) is a low cost, public health preventive measurement of the transmission of deadly viruses and bacteria on paper monies. Studies have demonstrated that over 375 deadly viruses and bacteria were found in paper monies and can be stably transferred from one individual to another. With the current global, pandemic, outbreak of the Corona Virus (COVID-19), thousands of people are dying and millions are placed on quarantine, China, the epic center of the virus, ordered their National Bank to manually disinfect their paper monies to prevent the spread of the CORVID-19) as monies was found to be a vector for transmission of the deadly virus and diseases.

In the case of banknotes, unlike coins, they are contaminated with contaminants such as various bacteria and dust, and cause bacterium transmission to users who use the banknotes. In addition, the paper money is not stored in a place for a long time but is circulated through the hands of many people. In the process, the paper money is contaminated with various bacteria and various pathogenic microorganisms to cause various skin diseases and respiratory diseases as well as allergic diseases And serve as mediators. As a result, the elderly or children who are weak in immunity are inflicted with diseases, and also there is a problem that the persons handling banknotes at banks are damaged.

On the other hand, in places such as banks, a large number of banknotes are to be quickly calculated and processed. Therefore, the banknote counter is used to calculate the number of banknotes. As described above, various kinds of bacteria and dust It is impossible to sterilize contaminants such as water.

There are multiple patents that can be seen in prior art. For instance, a Sterilizing bill counter bearing Chinese patent 2,755,684Y is issued to Chinese inventor. The patent discloses a utility model relates to a sterilizing banknote counting device which comprises a main body of the banknote counting device, and oxybromide generators or a sterilizer arranged in the main body of the banknote counting device, wherein the sterilizer is composed of an ultraviolet lamp tube and a metal sleeve coated with nanometer level Titanium dioxide film. The utility model adopts the structure that the oxybromide generators or the sterilizer composed of the ultraviolet lamp tube and the metal sleeve coated with the nanometer level Titanium dioxide are arranged in the main body of the banknote counting device. Therefore, hydroxyl radicals (—OH) are generated in the hydrosphere of the air by the active oxygen generated by the oxybromide generators, or by the ultraviolet rays with the wavelength as 253.7 nanometers under the catalysis action of Titanium dioxide. The hydroxyl radicals have strong oxidizing power, so that the bacteria in the air or the viral protein can be curdled quickly and lose activity. The goals of sterilization and disinfection can be achieved.

Another patent on a bill sterilizer storage type of a counting machine bearing Korean patent 1,015,45608B1 is issued to Korean inventor. The present invention relates to a bill sterilizer accommodating a counting machine. The bill sterilizer of the present invention comprises: a counting machine (100) having a bill input unit (120), a bill number sensing unit and a bill output unit (140); a main body having an opening at one side to have an accommodation space (210) inside, where the counting machine (100) is accommodated; a tray (300) arranged on the bottom surface of the accommodation space (210) to enter into the opening while moving back and forth via a rail (320) and have the counting machine (100) placed on the upper surface; an ion generator (400) located at the wall of the accommodation space (210) to spray cluster anions having a sterilizing function toward bills; a door (500), whose top end is fixed to a winding reel (520) connected to the main body over the opening to be capable of rotating and have an elastic force and is wound and which blocks the opening in being unwound; and a door locking portion (600) disposed on the front side of the main body (200) under the opening to fix the door (500). According to the present invention, the entire counting machine for measuring the number of bills is disposed and fixed inside the main body for sterilizing bills and then cluster anions are supplied to the inside of the main body, thereby enabling the cluster anions to be evenly sprayed to the surfaces of bills, and consequently removing contamination sources present on bills.

Another patent on Currency sterilization apparatus bearing US patent 2,011,0253563A1 is issued to Edward Goldman. The patent is on a standalone or retrofitted cash box that incorporates inner mounted ultra violet light sterilization and aerosol disinfectant release systems comprising automated actuation upon closing of the drawer, to reduce the amount of germs and bacteria found on currency both paper and coins at points of currency transaction. The retrofitted cash drawer component of the system comprises a perforated design to allow for greater surface exposure and currency desiccation quality.

Another patent on Sterilizer for paper bank note bearing Chinese patent 2,741,570Y is issued to Chinese inventor. The utility model provides a sterilizer which can sterilize not only one single piece of papery money but also stacks of papery money. An ozonizer, a container and a mechanical pump are orderly connected. The sterilizer is provided with two valves which can make one end of the container be connected with the ozonizer and the other end be connected with the mechanical pump. The stacks of papery money are arranged in the container. The air in the container is pumped through the mechanical pump. The ozone produced by the ozonizer sterilizes the papery money in the container. The sterilize has simple structure, little investment and good sterilizing effect.

Another Chinese patent 2,629,705Y on Banknote or paper bill sterilizing-disinfecting instrument is issued to Chinese inventor. The utility model discloses a sterilizing disinfector for paper money or paper bills, comprising two major parts, namely a mechanical drive and a sterilizing/disinfecting device. A plurality of methods, such as disinfecting solution, high temperature, and ultraviolet rays, are applied for complete and fast disinfection and sterilization purposes. After disinfection, the paper money or bill is dried and drawn without damage. This instrument can be used on-line with a cash counter. Disinfection and cash counting can be carried out at the same time. Disinfection and sterilization of paper money or bills in places where the paper money and bills are relatively concentrated can greatly reduce the chances that infectious diseases can be spread through paper money and bills.

Another patent on Sterilizing coin inlet arrangement bearing Chinese patent 2,514,433Y is issued to Chinese inventor. The utility model discloses a sterilizing cashier's machine which comprises an input device, a display, a casing, a currency storage bin and a print device, wherein the display device is directly placed on the top of the casing, at the same time the display device is electrically connected with the input device and an output device, the currency storage bin is slidably arranged in the interior of the casing, the top of the inner side of the casing is provided with a sterilizing device. The sterilizing device is an ultraviolet light generator which is directly fixed on the inner side upper wall of the casing through four bracing frames, the ultraviolet light generator is electrically connected with a power source through a directing line and a plug, at the same time, the ultraviolet light generator is electrically connected with a switch which is arranged on the outer wall of the casing. The sterilizing cashier's machine of the utility model can realize the sterilizing treatment for the circulating mediator which has fastest circulation and maximum range, thereby ensuring the health of human body.

Another Anti-Microbial Cash Drawer bearing US patent 2,015,0306263A1 is issued to Richard D. Yanke. The patent discusses system, methods and apparatus for efficiently and cost-effectively preventing the transfer of microbes, such as, for example, bacteria and viruses seamlessly during commercial transactions typically occurring at a full-service check-out station in a large retail store. The invention further considers the application of the methods and apparatus in other industrial disciplines where, for example, medical equipment and surgical tools may be susceptible to cross-contamination of microbes. One preferred embodiment incorporates a slidable drawer within a cash box where currency is stored, taken, or added randomly over a period of time. Especially designed lamps allow for a rapid switching means to control the time period for activating or deactivating lamps, thus regulating UV exposure. Exposure time with UVC and the orientation of the UV lamps provides complete germicidal decontamination within seconds.

Another patent on Sterilizing money safe bearing Chinese patent 2,514,089Y is issued to Chinese inventor. The patent discloses a disinfecting cash box, which includes a box body, a box door and a door lock, wherein, the box body is divided into upper and lower parts, the lower part is a box body used to store money of high par values, which is provided with a separated door, and the upper part is a box body used to separately store notes of different par values, the top of which is hinged with the box door. Four brackets are arranged on the inner sidewall of the box body of the disinfecting cash box, and an ultraviolet light generator firmly arranged on the four brackets is electrically connected with a power supply via a wire and a plug and electrically connected with a switch arranged on the outer sidewall of the box body as well. The disinfecting cash box of the utility model can sanitize the circulating intermediaries which circulate quickly and widely, which ensures the human health.

A U.S. Pat. No. 1,036,3328B2 on anti-microbial cash drawer is issued to Richard D. Yanke. The invention relates to a system, methods and apparatus for efficiently and cost-effectively preventing the transfer of microbes, such as, for example, bacteria and viruses seamlessly during commercial transactions typically occurring at a full service check-out station in a large retail store. The invention further considers the application of the methods and apparatus in other industrial disciplines where, for example, medical equipment and surgical tools may be susceptible to cross-contamination of microbes. One preferred embodiment incorporates a slide able drawer within a cash box where currency is stored, taken, or added randomly over a period of time. Especially designed lamps allow for a rapid switching means to control the time period for activating or deactivating lamps, thus regulating UV exposure. Exposure time with UVC and the orientation of the UV lamps provides complete germicidal decontamination within seconds. A caddy containing natural botanical cleaning components, method of use and a microbe testing method are described to provide retail stores, hospitals and restaurants a highly efficient and cost-effective means to reduce or eliminate microbes on store surfaces.

A Korean patent 2,003,0000280A on money cleaner is issued to Korean inventors. The patent provides a disinfector for notes which sprays perfume on the notes to disinfect the notes and presses the notes by a heating roller to show the effect of ironing. CONSTITUTION: The disinfector for the notes comprises the parts of: a shelf-typed fixer for the notes (11) which puts the notes; a supplier (12) which carries the notes by pieces; a notes carrier which carries the notes entered from the supplier; the disinfector (14) which disinfects the notes carried by the notes carrier; a drier (15) which dries the disinfected notes; a supplier for perfume which sprays perfume into the notes passed through the drier; a reception box (17) which receives the notes passed through the supplier for perfume; a notes counter which counts the number of the notes during carrying process; and a controller which controls the entire system.

Another patent on Disinfecting box for counting money bearing Chinese patent 2,168,737Y is issued to Chinese inventor. The utility model belongs to a disinfecting box for manually counting money which is composed of a disinfecting liquid reserve box, a self-suction tube, a honeycomb-shaped cylinder, non-woven fabric, antisepsis fabric, an outer cover, a liquid feeding hole and a seal cap. When counting the money artificially, the disinfecting box for manually counting money has the functions of preventing the stickiness of hands and sterilizing the hands. The disinfecting box for manually counting money is an ideal tool for preventing the bank notes from the spreading diseases to staffs of banks, financial accounting departments, and commercial trade.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease of use ability of these assemblies is also questionable.

It has to be noted that the current invention proposes an assembly with multiple advancement of currency container and dispenser which disinfect and kill germs from the currency. Unlike the conventional assemblies, the proposed Money Disinfectant is created with a dry heating element and with an Ultraviolet (UVC) light designed to kill germs, viruses and bacteria. Unlike chemical approaches to water disinfection, UV light provides rapid, effective inactivation of microorganisms through a physical process. When bacteria, viruses and protozoa are exposed to the germicidal wavelengths of UV light, they are rendered incapable of reproducing and infecting. The proposed assembly is equally effective for paper money and coins.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a reusable and cleanable container that relates to a sterilization apparatus for sterilization of paper money and coins.

It is another objective of the invention to provide an easy to use disinfectant assembly whom used is increasingly agreeable for the banking professionals and normal users.

It is further the objective of the invention to provide an advance level of assembly where a standalone, or retrofitted cash box that incorporates interior mounted ultra violet light sterilization and integrally disposed disinfectant release systems comprising automated actuation upon closing of the cash drawer, in order to reduce the amount of germs and bacteria found on currency both paper and coins at points of currency transaction (POS point of sale).

It is also the objective of the invention to provide a device which includes design modifications for easy repair and maintenance. A modular design provides for a rapid and simple manufacturing process.

It is also the objective of the invention to provide a device where the invention provides germicidal protection, by retro fitting current cash drawers with a UV system that significantly reduces or eliminates the exchange of microbes occurring with the transfer of currency.

It is further the objective of the invention to provide a detachable assembly that can be installed and carried easily.

It is another objective of the invention to provide a device that is crafted from very firm, material to give user the assembly which can work for long term without any effect on its output effectiveness.

It is moreover the objective of the invention to provide assembly which acquires less space and is easy to install and simple to use.

A still further object is to provide an assembly that is economical in cost to manufacture.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

FIG. 1 discloses a metal box, which contains a UVC Light lamp and a heating element with temperature controls.

FIG. 2 discloses a top view of the Money Disinfectant (MD) where multiple control tabs can be seen as per preferred embodiments of the invention.

FIG. 3 discloses another bottom view of assembly where UV light and heating element of the Money Disinfectant (MD) can be seen as per preferred embodiments of the invention.

FIG. 4 discloses another complete view of assembly where Money Disinfectant (MD) can be used as an attachment to a typical money counting machine as per preferred embodiments of the invention.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

According to the World Health Organization viruses are inactivated at temperatures between 60 and 65 degrees Celsius. The temperature to kill bacteria differs with the bacterial habitat. Although most bacteria do not live above 102 degrees Fahrenheit. At 102 degrees Fahrenheit most bacteria can no longer reproduce.

Temperature of 165 Fahrenheit to 250 Fahrenheit will kill most of the germs and yet some viruses can't be killed. This is because they don't have structure that can be destroyed by heat, and an Ultraviolet Light is added to kill them. The exact temperatures that kill germs, however, depend on the microbe and how long it stays in the heat.

Using the above information, the Money Disinfectant was created with a dry heating element and with an Ultraviolet (UVC) Light designed to kill germs, viruses and bacteria. Unlike chemical approaches to water disinfection, UV light provides rapid, effective inactivation of microorganisms through a physical process. When bacteria, viruses and protozoa are exposed to the germicidal wavelengths of UV light, they are rendered incapable of reproducing and infecting.

Research has further proved that UV Light can kill viruses, including the Flu. It has been known that broad spectrum of Ultraviolet C (far-UVC) light has the power to kill bacteria and viruses by breaking molecular bonds. The average bacterium will be killed in ten seconds at a distance of six inches from the light in a Ultraviolet germicidal fixture. The dosage, a product of UV light intensity and exposure time, is usually measured in microjoules per square centimeter, or equivalently as microwatt seconds per square centimeter Dosages for a 90% kill of most bacteria and viruses range from 2,000 to 8,000.

UVC light is part of the ultraviolet light spectrum and emits a high frequency of UV light that makes it extremely effective at killing bacteria, viruses, mold and other pathogens. Killing bacteria with UV light requires the use of germicidal wavelengths of 185-254 nanometers (nm).

The effectiveness of the UVC light is proven as the use of UV sterilization, or ultraviolet germicidal irradiation, has been found to be extremely effective. Sources of UV sterilization can kill over 99% of viruses, bacteria, and fungi in an extremely short amount of time. The time period to disinfect with the UV light can have efficient inactivation of bacteria up to a distance of eight feet on either side and exposure time of 30 minutes is adequate.

Also, the safety of the UV light has been proven in that UV Light that Is safe for humans but bad for Bacteria and Viruses. Scientists have known for decades that broad-spectrum UVC light, which has a wavelength of between 200 to 400 nanometers (nm), is highly effective at killing bacteria and viruses by destroying the molecular bonds that hold their DNA together.

The Money Disinfectant (MD) invention as per preferred embodiments functions using UV light and by killing over 100 deadly viruses, bacteria and germs causing illnesses, found on paper monies, by applying heater and/or Ultraviolet light (UV).

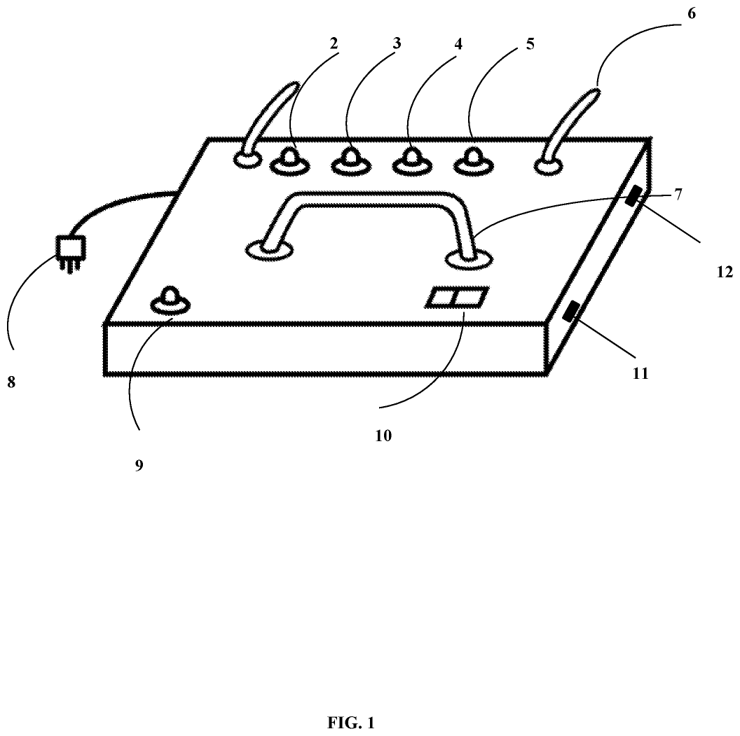

FIG. 1 illustrates the metal box, which contains a UVC Light lamp and a heating element with temperature controls. The assembly consist of following parts Metal Box (1), Ultraviolet timer control knob (2), UV intensity control knob (3), Heat temperature Control Knob (4), heat timer Control Knob (5), clip to attach to money counting machine (6), Unit Handle (7), Power Cord (8), On/Off Indicator light (9), On/Off switch (10), UV light (11) and heating element (12).

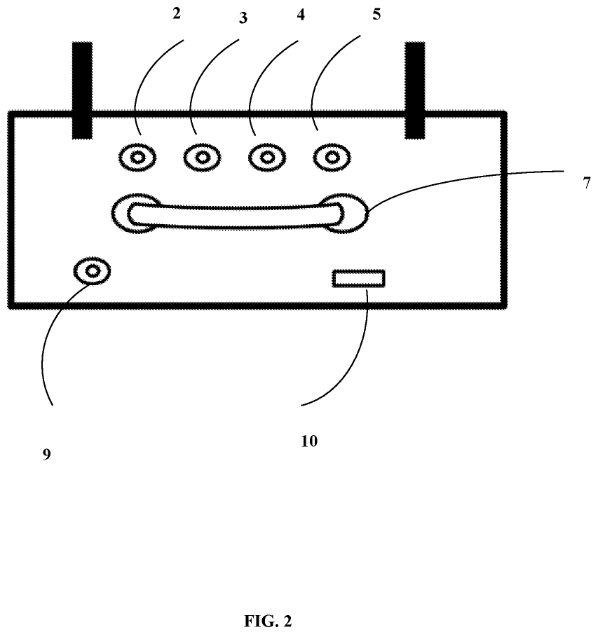

FIG. 2 discloses a top view of the Money Disinfectant (MD) where multiple control tabs Ultraviolet timer control knob (2), UV intensity control knob (3), Heat temperature Control Knob (4), heat timer Control Knob (5) Unit Handle (7), On/Off Indicator light (9), On/Off switch (10) can be seen as per preferred embodiments of the invention.

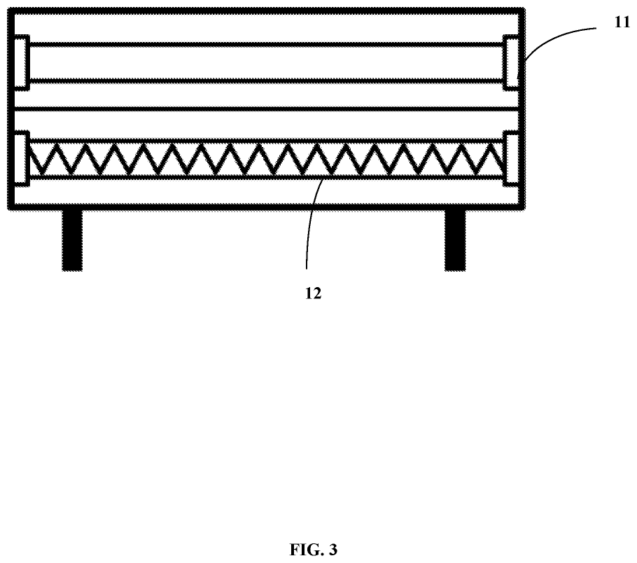

FIG. 3 discloses another bottom view of assembly where UV light (11) and heating element (12) of the Money Disinfectant (MD) can be seen as per preferred embodiments of the invention.

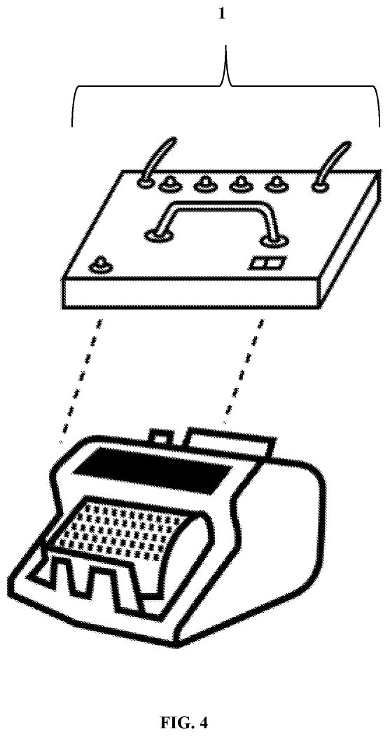

FIG. 4 discloses another complete view of assembly where Money Disinfectant (1) can be used as an attachment to a typical money counting machine as per preferred embodiments of the invention.

The metal box (1) dimension as per its illustrative embodiments is 8″ of length by 6″ in width by 2″ high top of the box and has a handle attached to it. The anterior portion of the box has on/off switch (10) where the on/off indicator light (9), turns green when unit is On. The top of the box has also a Heat timer control knob (5) to measure amount of time that heat is applied, from 0-60 minutes.

The top portion of the metal box also has heat temperature control knob (4) to control the amount of heat being applied in Fahrenheit and Celsius degrees, ranging from 102-250 degrees. The assembly further has Far-UVC Light Timer control knob to measure farUVC light exposure time, ranging from O to 10 minutes. The far-UVC Light intensity control knob (3), measure in nanometers (nm) from 200-400 or in micro watts. The assembly has further a Power cord on the right side of assembly and can also be battery operated.

The top of the box has 2 clips which are used to attach the unit to other equipment such as a typical money counter machine, cash registers, ATMs, etc.

The assembly contain far-Ultraviolet light bulb (2) with electrical connections. The UV light range from 200-400 nanometer (nm). The UV light provides rapid, effective inactivation of microorganisms through a physical process. When germs, bacteria, viruses and protozoa are exposed to the germicidal wavelengths of UV light, they are rendered incapable of reproducing and infecting. Research further shows that it takes one minute to kill deadly viruses and bacteria using the UV light.

The assembly as per further embodiments contains a dry heating element (3) with a timer, control knob and a temperature control knob: the timer knob is (O to 10 minutes); and the temperature control knob has a range of temperature capacity of 102 to 250 degrees Fahrenheit, 38.9 and 121.1 degree Celsius.

The UV Light element contain a bulb, far-ultraviolet light (UVC) that is controlled by 2 knobs to measure time and intensity. The timer knob (4) ranges from (0-10 minutes); where the exposure intensity knob measures the intensity exposure and UV light from 200-400 nanometers (nm) or microwatt UV Light which is safe for humans. The Timer (4) where the time range from 0-10 minutes allows the user to set time as required.

The temperature control knob (5) has a ranging temperature capacity of 102 to 250 degrees Fahrenheit, 38.9 and 121.1 degree Celsius. The temperature to kill bacteria differs with bacterial habitat. At 102 degrees Fahrenheit most bacteria can no longer reproduce.

The assembly can be turned on or off when required. It has handles (7) which allows ease of portability. The clips (6) are used to attach unit to another machine, such as money counting machines, cash registers, ATMs, etc. The assembly can be plugged into electrical outlet for Power (8).

The device as per its further embodiments is crafted from extra firm material to give the ultimate support needed for optimal performance. The device is designed to be made of stainless steel to withstand long time usage. It is also small, lightweight, and easy to use which makes it the superior choice for professionals and for every common user.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

I: a currency purification and distribution system comprising:
a cash box system where the outer casing has
UV timer control knob;
UV intensity control knob;
heat temperature control knob;
heat timer control knob;
heating element;
on/off indicator;
clips to attach to money counting machine and power cord.

II: The currency storage, purification and distribution system wherein said ultraviolet light administration system and said disinfectant system are triggered upon turning on/off the indicator buttons.
the currency purification system of claim 1 wherein said ultraviolet light administration system comprises an ultraviolet light panel comprising far-Ultraviolet light bulb with electrical connections;
the currency purification system of claim 1 wherein said system has timer control knob to control time from 1-10 minutes;
the currency purification system of claim 1 wherein said system has temperature control knob having a range of temperature capacity of 102 to 250 degrees Fahrenheit, 38.9 and 121.1 degree Celsius;
the currency purification system of claim 1 wherein said system has exposure intensity knobs measure the intensity exposure and UV light from 200-400 nanometers or microwatt;
the currency purification system of claim 1 wherein said system has handle for ease in portability;
the currency purification system of claim 1 wherein said system has clips to attach to money counting machine if required and
the currency purification system of claim 1 wherein said system has power cord to meet energy requirements.
